(19) United States
(12) Patent Application Publication
(10) Pub. No.: US 2014/0330031 A1
(43) Pub. Date: Nov. 6, 2014

(54) RESIST FOR ELECTRON BEAM AND OPTICAL LITHOGRAPHY

(71) Applicant: Council of Scientific & Industrial Research, New Delhi (IN)

(72) Inventors: Mohan Gopalkrishna Kulkarni, Pune (IN); Dadasaheb Vitthal Sangave, Pune (IN)

(21) Appl. No.: 14/363,240

(22) PCT Filed: Dec. 6, 2012
(86) PCT No.: PCT/IN2012/000792

§ 371 Date: Jun. 5, 2014

(30) Foreign Application Priority Data
Dec. 5, 2011 (IN) 3496/DEL/2011

(51) Int. Cl. G03F 7/027; C07C 41/01; C07D 303/28; C07C 43/23

(52) U.S. Cl. CPC G03F 7/027; C07C 43/23; C07C 41/01; C07D 303/28
USPC 549/560; 568/633

(57) ABSTRACT

The present invention describes a first generation dendrimers useful in lithography, comprising 1,3,5-trisbromo-methylbenzene as the core and dense, bulky, rigid units selected from trisphenol (1, 1, 1-tris-p-4-hydroxyphenyl ethane), bisphenol-A and 1,5-dihydroxy naphthalene units at the periphery, wherein the peripheral aromatic rigid molecules are connected to the central core through an ether linkage.

### TECHNICAL FIELD OF THE INVENTION

The present invention relates to a first generation dendrimer (FGD) comprising a core group and a peripheral moiety linked to the core wherein peripheral moiety has a functional group which has been chemically modified for a resist application.

### BACKGROUND AND PRIOR ART OF THE INVENTION

Potential applications of dendrimers are based on their molecular uniformity, multifunctional surface and presence of internal cavities. These properties make dendrimers suitable for a variety of high technology applications in in vitro diagnostics; as contrast agents for magnetic resonance; in the targeted delivery of therapeutic agents; as coating agents to protect or deliver drugs to specific sites in the body or as time-release vehicles for biologically active agents, as carriers in gene therapy and industrial applications such as catalysts or as resists in electron beam as well as optical lithography.

A photoresist is a light-sensitive material used in industrial processes, such as photolithography and photoengraving to form a patterned coating on a surface. The resists can be classified into two types namely negative resists and positive resists. A positive resist is one in which the portion of the photoresist that is exposed to light becomes soluble in the photoresist developer. The portion of the photoresist that is unexposed remains insoluble in the photoresist developer. On the contrary, a negative resist is a type of photoresist in which the portion of the photoresist that is exposed to light becomes insoluble in the photoresist developer. The unexposed portion of the photoresist is dissolved by the photoresist developer.

Use of low molecular weight resists such as dendrimers in electron beam lithography (EBL) is well known in the art. Most of the low molecular weight resists and dendrimers used for EBL contain free hydroxyl groups and reactive furan rings or Powderlink1174 (tetrakis(methoxylmethyl)-glycoluril. Crosslinking of these systems during post exposure bake (PEB) results in the generation of small gas molecules such as water vapor and methanol, which may lead to pattern deformation especially at lower feature sizes. It is therefore desirable to avoid crosslinking reactions which result in the release of low molecular weight products. The most widely used photoresist systems are based on the crosslinking of epoxy resins.

The crosslinking of negative photoresist containing epoxide groups is caused by the irradiation of a photoacid generator, which generates a reactive oxygen species that reacts with the epoxide moiety on a neighboring polymer chain, resulting in the formation of a cross-link and corresponding propagating cation. This chain reaction leads to highly sensitive resists with high cross-linking efficiencies (Argitis et al., 1998).

Negative photoresists based on the epoxy resins are used extensively in the electronics industry as they offer a unique combination of properties like high strength, thermal stability, moisture resistance, chemical and corrosion resistance, adhesion and requisite mechanical as well as electrical properties.

SU-8 is a negative tone epoxy photoresist, which provides good lithographic performance. It is extensively used for applications in lithography and for molding and packaging, however it suffers from certain limitations. The spin-coating of both thick and thin layers of SU-8 resist often does not result in homogenous films. Debonding after post-baking and development results from poor adhesion to substrate. Also cracking at the corners of the microstructures has been reported.

Conventional epoxy resin (SU-8) contains eight epoxide groups and has T_{g} 50° C. before crosslinking, which is enhanced to 200° C. or more when fully cross linked (Balakrishnan et al., 2006; Feng and Farris 2003). It is recognized that for negative photoresists, increase in T_{g} with crosslinking is desirable since increase in difference in T_{g} between the exposed and unexposed areas results in greater solubility difference, which can be expected to result in better resolution and contrast as well as low line edge roughness (LER). Bilenberg et al (2006) reported 24 nm features at a pitch 1:12.5 nm at 19.9 μC/cm² using 100 kV beam energy. However, these resins suffer from brittleness, resulting from high cross link density, higher shrinkage and stress generated during crosslinking, which results in cracks and adhesion problems.

Apart from SU-8 negative tone photoresist, a number of polymeric negative photoresists have been used in the past. However, lithographic performance of polymeric resist is affected by many factors like high molecular weights, broad molecular weight distribution, chain entanglement which results in poor performance especially irregularity of patterns.

Efforts are therefore underway to develop newer epoxy based resist for EBL. The negative resist based on calix[4]arene bearing epoxide was reported by Sailer et at (2004). The resist was processed by EBL at 80 μC/cm² and 30 kV beam energy. 25 nm lines and 35 nm dots were resolved when pitch was set more than 150 nm. But the contrast was low (2.1).

The crosslink density can be manipulated by the choice of the peripheral moiety and number of hydroxyl or epoxide groups. Haba et al., (1999) reported dendrimer based on Calix[4]resorcinarene containing 16-hydroxyl groups at the periphery for optical lithography.

Since the solubility of this dendrimer in aqueous tetramethyl ammonium hydroxide solutions was very high, only a very dilute solution could be used as a developer. To overcome this problem another dendrimer containing 6-hydroxyl groups at the periphery was used (Kamimura et al., 2005). Decreasing peripheral hydroxyl groups from 16 to 6, enabled reduce the feature size from 3 μm to 1 μm.

Inspite of these developments there is a need in the art to design photoresists that can overcome the limitation of the dendrimers reported in the past for the lithographic application and enable fabrication of patterns upto 30 nm and a pitch 1:1, that can be used for EBL applications.

### ABBREVIATIONS USED IN THE INVENTION

G1: 1,3,5-trisbromo-methylbenzene
Bis: bisphenol-A
Tris: Trisphenol (1, 1, 1-tris-p-4-hydroxyphenyl ethane)
Dhn: 1,5-dihydroxy naphthalene
t-BOC: Di-tert-butyl dicarbonate
EBL: Electron beam lithography
FGD: First generation dendrimers

### BRIEF DESCRIPTION OF THE DRAWINGS

FIG. 1 depicts FGD s containing peripheral functional groups

FIG. 2 depicts Relative resist thickness vs. exposure dose for the FGDs: A) 5 wt % PAG, B) 10 wt % PAG

FIG. 3 depicts SEM images of G₁-Tris-epoxide used for sensitivity and contrast calculation: A) 5 wt %; B) 10 wt. % PAG (perfluoro-1-butanesulfonate).

FIG. 4 depicts Images of patterns of G₁-Tris-epoxide used for Line edge roughness (LER) calculation

FIG. 5 depicts Optical microscopy images of the resist A) SU-8, B) G₁-Tris-epoxide, C) G₁-Bis-epoxide and D) G₁-Dhn-epoxide

FIG. 6 depicts SEM images of A) G₁-Tris-epoxide and B) G₁-Bis-epoxide EBL pattern. Line thickness 500, 200, 100 and 50 nm bottom upward.

FIG. 7 depicts A) SEM image of 50 nm lines at 1:2 nm pitch for G₁-Tris-epoxide; B) 50 nm lines at 1:10 nm pitch for G₁-Dhn-epoxide.

FIG. 8 depicts 30 nm lines at pitch A) 1:10, B) 1:2, (C) 1:1, G₁-Tris-epoxide

FIG. 9 depicts 30 nm lines at pitch A) 1:10, B) 1:2, (C) 1:1, G₁-Bis-epoxide

FIG. 10 depicts the etch rate was calculated by the difference of the thickness before (C) and after (F) etching.

FIG. 11 depicts T_{g} curve for G₁-Bis-t-BOC

FIG. 12 depicts T_{g} curve for G₁-Tris-t-BOC

FIG. 13 depicts T_{g} curve for G₁-Dhn-t-BOC

FIG. 14 depicts Sensitivity curve for the positive dendrimeric resist

FIG. 15 depicts SEM image of cross section of FGD G₁-Tris-t-BOC 80 used for sensitivity and contrast

FIG. 16 depicts SEM images of 100 nm (A, B, C), 50 nm (D, E) and 30 nm (F, G, H) with varying pitch patterns obtained using FGD G₁-Tris-t-BOC 80 with EB lithography, dose 50 μC/cm²

FIG. 17 depicts SEM images of 100 nm (A), 50 nm (B, C) and 30 nm (D, E, F) with varying pitch patterns obtained using FGD G₁-Bis-t-BOC 80 with EB lithography, dose 60 μC/cm²

FIG. 18 depicts SEM images of 100 nm (A) 50 nm (B) with 1:2 pitch patterns obtained using FGD G₁-Dhn-t-BOC 80 with EB lithography, dose 80 μC/cm²;

FIG. 19 depicts SEM images of 100 nm (A) 50 nm (B) with 1:5 and 1:10 pitch negative tone patterns obtained using FGD G₁-Tris-t-BOC 80 with EB lithography, dose 50 μC/cm²

### SUMMARY OF THE INVENTION

In accordance with the above, in one aspect, the present invention provides first generation dendrimers having 1,3,5-trisbromo methylbenzene as a core using different rigid groups such as bisphenol, trisphenol (1, 1, 1-tris (4-hydroxyphenyl) ethane) and 1, 5 dihydroxy naphthalene as peripheral groups to manipulate T_{g} from 38 to 85° C.

According to the invention, the choice of peripheral groups allows number of the peripheral hydroxyl groups to be varied. In case of bisphenol and naphthalene based dendrimers three hydroxyl groups are present on the periphery, while in the case of 1, 1, 1-tris (4-hydroxyphenyl) ethane) six hydroxyl groups are present on the periphery. Polar ether linkage is introduced between the core and the peripheral groups to enhance adhesion.

Terminal hydroxyl groups on the periphery were subsequently reacted with epichlorohydrin to obtain epoxy function and yield a negative photoresist or conjugated with t-BOC to yield positive photoresists viz. G₁-Tris-t-BOC, G₁-Bis-t-BOC and G₁-Dhn-t-BOC.

In another aspect, the FGDs prepared according to the invention are evaluated for their applications as negative as well as positive tone electron beam resist.

### BRIEF DESCRIPTION OF THE INVENTION

In an embodiment the present invention provides a first generation dendrimers comprising 1,3,5-trisbromo-methylbenzene as the core and dense, bulky, rigid units selected from Trisphenol (1, 1, 1-tris-p-4-hydroxyphenyl ethane), bisphenol-A and 1,5-dihydroxy naphthalene units at the periphery, wherein the peripheral aromatic rigid molecules are connected to the central core through an ether linkage.

In another embodiment the present invention provides a first generation dendrimers wherein the dendrimers based on trisphenol (1, 1, 1-tris-p-4-hydroxyphenyl ethane) comprise six hydroxyls at the periphery.

In another embodiment the present invention provides a first generation dendrimers wherein the dendrimers based on bisphenol-A and 1,5-dihydroxy naphthalene comprise three hydroxyl at the periphery.

In yet another embodiment the present invention provides a first generation dendrimers wherein the dendrimers are further conjugated with either epoxide or with tert-BOC to obtain negative or positive photoresists respectively.

In yet another embodiment the present invention provides a first generation dendrimers wherein the negative photoresists are selected from the group consisting of G₁-Tris-epoxide, G₁-Bis-epoxide and G1-Dhn-epoxide.

In yet another embodiment the present invention provides a first generation dendrimers wherein positive photoresists are selected from the group consisting of G₁-Tris-t-BOC-100, G₁-Tris-t-BOC-80, G₁-Tris-t-BOC-60, G₁-Tris-t-BOC-50, G₁-Bis-t-BOC-100, BOC-80, G₁-Bis-t-BOC-60, G₁-Bis-t-BOC-50, G₁-Dhn-t-BOC-100, G₁-Dhn-t-BOC-80, G₁-Dhn-t-BOC-60 and G₁-Dhn-t-BOC-50.

In yet another embodiment the present invention provides a first generation dendrimers wherein the yield of positive photoresist is obtained in order of ≧65%.

In yet another embodiment the present invention provides a first generation dendrimers wherein glass transition temperature of positive photoresists is in the range of 45° C. to 130° C.

In yet another embodiment the present invention provides a first generation dendrimers wherein the molecular weights of the positive photoresists are in the range of 700 to 1700.

In yet another embodiment the present invention provides a first generation dendrimers wherein (G₁-Tris-t-BOC 80), (G₁-Bis-t-BOC 80) and (G₁-Dhn-t-BOC 80) having contrast γ=2.50, γ=2.09 and γ=1.66 respectively in presence of 10 wt % PAG.

In a further embodiment the present invention provides a first generation dendrimers wherein (G₁-Tris-t-BOC 80), (G₁-Bis-t-BOC 80) and (G₁-Dhn-t-BOC 80) having sensitivity 50 μC/cm²; 60 μC/cm² and 80 μC/cm² respectively at 20 keV electron beam acceleration.

In a further embodiment the present invention provides a process for synthesis of negative photoresists according to claim 3, comprises reacting phenols/naphthols with 1,3,5-tris-bromomethylbenzene in the presence of potassium carbonate in DMF; conjugating the peripheral hydroxyls with epichlorohydrin using KOH as a base and PEG-400 as a phase transfer catalyst.

In a further embodiment the present invention provides a process wherein the molecular weights of the negative photoresists are in the range of 750-1400.

In a further embodiment the present invention provides a process wherein the glass transition temperature of negative photoresists is in the range of 60° C. to 90° C.

In a still further embodiment the present invention provides a process wherein the yield of negative photoresist is obtained in the order of >90%.

In a still further embodiment the present invention provides a process wherein the negative photoresists with 10 wt % PAG content shows a sensitivity of 35 μC/cm².

In a still further embodiment the present invention provides a process wherein negative photoresists having resist etching rate in the range of 0.23 to 0.30.

In accordance with the need, the instant invention provides first generation dendrimers starting with 1,3,5-trisbromo-methylbenzene as the core and dense, bulky, rigid units selected from Trisphenol (1, 1, 1-tris-p-4-hydroxyphenyl ethane), bisphenol-A and 1,5-dihydroxy naphthalene units at the periphery. The peripheral aromatic rigid molecules are connected to the central core through an ether linkage.

Accordingly, in one embodiment, first generation dendrimer (FGD) based on trisphenol has six hydroxyls at the periphery.

In another embodiment, first generation dendrimers based on bisphenol-A and 1,5-dihydroxy naphthalene have three hydroxyl at the periphery. This variation at the periphery helps to vary crosslink density. Also, T_{g} could be manipulated by the choice of the peripheral aromatic group.

In another embodiment, the terminal hydroxyls in both the cases were reacted with epichlorohydrin to yield negative resists which were processed by EBL. These negative tone e-beam resists can be used to fabricate structures upto 30 nm width at a pitch 1:1.

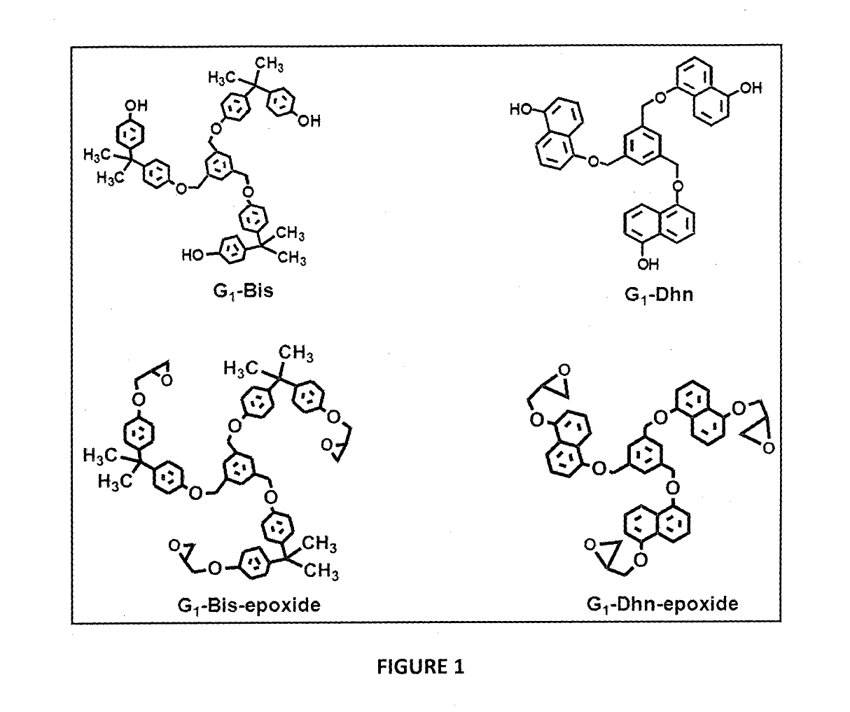

Accordingly, in the instant invention ether functionality on the benzene core as shown in FIG. 1. incorporated. The first generation dendrimers were synthesized by reacting phenols and/or naphthols with 1,3,5-tris-bromomethylbenzene in the presence of potassium carbonate in DMF. The peripheral hydroxyls were conjugated with epichlorohydrin using KOH as a base and PEG-400 as a phase transfer catalyst. The molecular weights of the FGDs are summarized in Table 1.

The molecular weights of the negative photoresists were found to be in the range of 750-1400; whereas the glass transition temperature of negative photoresists was measured in the range of 60° C. to 90° C.

Further the yield of negative photoresists synthesiszed by the said process was obtained in the order of >90%.

TABLE 1
Characterization of first generation dendrimers

| | | Molecular | | |
|---|---|---|---|---|
| Sr. No. | Dendrimers | weight | T_{g}° C. | T₅° C. |
| 1 | G₁-Tris-epoxide | 1370 | 80.00 | 348 |
| 2 | G₁-Bis-epoxide | 0967 | 38.47 | 326 |
| 3 | G₁-Dhn-epoxide | 0763 | 66.60 | 212 |
| 7 | G₁-Tris | 1033 | 139.00 | 273 |
| 8 | G₁-Bis | 0799 | 70.00 | 198 |
| 9 | G₁-Dhn | 0595 | 87.00 | 139 |

In another preferred embodiment, the terminal hydroxyls in all the three cases were conjugated with t-BOC using procedures reported in literature (Hansen and Riggs, 1998) to yield positive photoresists viz. G₁-Tris-t-BOC, G₁-Bis-t-BOC and G₁-Dhn-t-BOC. Degree of conjugation was controlled by controlling the amount t-BOC in the feed. The molecular weights of the FGDs are summarized in Table 2.

The molecular weights of the positive photoresists were found to be in the range of 700 to 1700, whereas glass transition temperature of positive photoresists was measured in the range of 45° C. to 130° C.

Further the yield of positive photoresists disclosed in Table 2 was obtained in order of ≧65%.

TABLE 2
Molecular weights, and T_{g} of dendrimers

| No. | Dendrimers | MW | T_{g}° C. |
|---|---|---|---|
| 1 | G₁-Tris-t-BOC-100 | 1633 | 119 |
| 2 | G₁-Tris-t-BOC-80 | 1488 | 125 |
| 3 | G₁-Tris-t-BOC-60 | 1355 | 130 |
| 4 | G₁-Tris-t-BOC-50 | 1318 | 128 |
| 5 | G₁-Tris | 1033 | 139 |
| 6 | G₁-Bis-t-BOC-100 | 1102 | 45 |
| 7 | G₁-Bis-t-BOC-80 | 961 | 58 |
| 8 | G₁-Bis-t-BOC-60 | 923 | 63 |
| 9 | G₁-Bis-t-BOC-50 | 902 | 68 |
| 10 | G₁-Bis | 799 | 70 |
| 11 | G₁-Dhn-t-BOC-100 | 895 | 126 |
| 12 | G₁-Dhn-t-BOC-80 | 777 | 118 |
| 13 | G₁-Dhn-t-BOC-60 | 753 | 116 |
| 14 | G₁-Dhn-t-BOC-50 | 724 | 116 |
| 15 | G₁-Dhn | 595 | 87 |

In yet another preferred embodiment, the first generation dendrimers prepared according to the invention have been evaluated for their negative as well as positive tone electron beam resist as well as negative for optical resist.

The resists based on 1, 1, 1-tris-p-4-hydroxyphenyl ethane and bisphenol-A evaluated as negative photoresist could resolve 30 nm lines at a pitch 1:1. The negative tone resists based on FGDs were evaluated for optical lithography. Reliable reproduction of structures with minimum feature size of 4.3 μm was possible using FGDs. An RIE process was used to check resist stability and the RIE results based on FGDs show greater stability compared to SU-8.

Further, low molecular weights FGDs varying in t-BOC functionality have been demonstrated to function as positive electron beam resist. Sensitivity of 50 μC/cm² and contrast γ=2.5 in electron beam lithography in thin layers (70 nm) with dendrimer based on trisphenol has been demonstrated. Patterning of 30 nm lines with 30 nm pitch on silicon substrate was achieved successfully.

### EXAMPLES

Following examples are given by way of illustration and therefore should not be construed to limit the scope of the invention.

### Example 1

### Preparation of G₁-Tris

Trisphenol 47 g (0.156 moles) was dissolved in 400 ml DMF, 97 g (0.702 moles) of anhydrous potassium carbonate was added and stirred for 30 min and maintained at 65° C., 2.8 g (7.8×10⁻³ moles) of 1, 3, 5-trisbromomethyl benzene in 20 ml DMF was added drop wise over 40 min. After the addition was complete, stirring was maintained for 12 h. DMF was recovered on rotary evaporator and reaction mixture was extracted with ethyl acetate. The solvent was evaporated and the G₁-Tris formed was purified by column chromatography, using petroleum ether and ethyl acetate (70:30 v/v) as elutant.G₁-Tris yield was 8.50 g (85%).

¹H-NMR spectrum of G₁-Tris in acetone-4 indicate complete disappearance of the peak at 4.50 ppm corresponding to methylene in 1, 3, 5-trisbromomethyl benzene and appearance of the peak at 5.11 ppm confirms formation of G₁-Tris. Peak at 9.28 ppm corresponds to the aromatic hydroxyl functionality of Trisphenol unit and at 7.52 ppm to the aromatic protons of central benzene unit, which confirms conjugation of trisphenol and benzyl groups. It was thus concluded that, all benzyl bromide groups were converted to corresponding hydroxyl groups of G₁-Tris.

### Example 2

### Preparation of G₁-Bis

25.5 g (0.112 moles) bisphenol-A, 47g (0.336 moles) of K₂CO₃ in 150 ml DMF and 2 g (5.6×10⁻³moles) of 1, 3, 5-trisbromomethyl benzene in 20 ml DMF were reacted as described above. G₁-Bis yield was 5.00 g (87%).

¹H NMR (DMSO-d₆): 8.19 δ(3H, -OH), 6.7-7.19 (24H, phenolic protons), 7.56 (3H, benzylic protons), 5.13 (6H, Ph-CH₂), 1.62 (18H, —CH₃).

### Example 3

### Preparation of G₁-Dhn

18 g (0.112 moles) 1, 5-Dhn, 47 g (0.336 moles) of K₂CO₃ in 150 ml DMF and 2 g (5.6×10⁻³ moles) of 1, 3, 5-trisbromomethyl benzene in 20 ml DMF were reacted as described above G₁-Dhn yield was 3.80 g (81%).

¹H NMR (DMSO-d₆): 9.10 δ(3H, —OH), 6.94-7.85 (18H, naphthalene ring protons), 7.88 (3H, benzylic protons), 5.42 (6H, Ph-CH₂).

### Example 4

### Preparation of G₁-Tris-epoxide

5 g (5×10⁻³ moles) G₁-Tris, 0.05 g polyethylene glycol (PEG-400) as a phase transfer catalyst and 50 ml epichlorohydrin were added to a 100 ml two necked flask equipped with a stirrer. The contents were heated to 70° C. for 1 h. To this reaction mixture dilute aqueous potassium hydroxide solution (KOH) 1.70 g (3×10⁻² moles) in 10 ml water was added drop wise over 40 min. After addition of alkali was complete, the reaction was continued at 60° C. for further 2 h. The reaction mixture was filtered and the organic phase was washed with water three times and dried over anhydrous sodium sulphate. Excess epichlorohydrin was recovered over rotary evaporator under vacuum to yield a semisolid mass, which was then precipitated from pet ether. The product was purified by dissolving it in THF and reprecipitated from pet ether to recover a colorless product. The yield of G₁-Tris-epoxide was 7.50 g (97%).

¹H-NMR spectrum of G₁-Tris-epoxide in CDCl₃ shows that peak at 9.28 ppm corresponding to the aromatic hydroxyl is replaced by peaks corresponding to epoxide ring protons (2.72-4.2 ppm), and confirms conjugation of epoxide groups.

### Example 5

### Preparation of G₁-Bis-epoxide

The compound was prepared by the same method used for G₁-Tris-epoxide using 2 g (2.5×10⁻³ moles) G₁-Bis, 0.02 g polyethylene glycol (PEG-400) as a phase transfer catalyst, 20 ml epichlorohydrin and potassium hydroxide 0.63 g (1.1×10⁻² moles) in 4 ml water. G₁-Bis-epoxide yield was 3.30 g (98%).

¹H NMR (CDCl₃-d₆); δ [ppm]: 6.79-7.15 (24H, phenolic protons), 7.44 (3H, benzylic protons), 5.04 (6H, Ph-CH2), 1.63 (18H, —CH3), 2.72 to 2.92 ppm (6H, multiplet, protons of methylene in the oxirane ring), 3.43 ppm (3H, multiplet, protons of methine in the oxirane ring), 3.89 to 4.19 ppm (6H, multiplet, protons of methylene connecting the phenoxy and the oxirane ring),

### Example 6

### Preparation of G₁-Dhn-epoxide

G₁-Dhn-epoxide was prepared from, 2 g (3.36×10⁻³moles) G₁-Dhn, 0.02 g polyethylene glycol (PEG-400) as a phase transfer catalyst and 20 ml epichlorohydrin and potassium hydroxide 0.85 g (1.5×10⁻² moles) in 5 ml water. G₁-Dhn-epoxide yield was 2.60 g (93%).

¹H NMR (CDCl₃); δ [ppm]: 6.79-7.15 (24H, naphthalene ring protons), 7.44 (3H, benzylic protons), 5.29 (6H, Ph-CH2), 2.83 to 2.99 ppm (6H, multiplet, protons of methylene in the oxirane ring), 3.47 ppm (3H, multiplet, protons of methine in the oxirane ring), 3.86 to 4.41 ppm (6H, multiplet, protons of methylene connecting the naphthalene and the oxirane ring).

### Example 7

### Preparation of G₁-Tris-t-BOC100

3 g (2.9×10⁻³ moles) of G₁-Tris and 3.2 g (2.6 10⁻² moles) DMAP were dissolved in 25 ml NMP and stirred for 20 min. A solution of di-t-BOC 5.7 g (2.6×10⁻² moles) in 10 ml NMP was then added drop wise to the solution at 0-5° C. After complete addition, the mixture was stirred for 24 h at room temperature. The product was precipitated from methanol. White powder was obtained after drying the product in a vacuum oven at 45° C. G₁-Tris-t-BOC-100 was obtained in good yield, 3.7 g (78%).

¹H NMR in CDCl₃ shows that after conjugation with t-BOC peak at 9.28 ppm corresponding to aromatic hydroxyls, was replaced by the peak at 1.54 ppm corresponding to the methyl protons of t-BOC units. All phenolic hydroxyl groups at the periphery were converted to corresponding t-BOC group.

### Example 8

### Preparation of G₁-Tris-t-BOC-80

The compound was prepared by same method used for G₁-Tris-t-BOC100 dendrimer from, 3 g (3×10⁻³ moles) of G₁-Tris and 2.64 g (0.216 moles) DMAP, 25 ml NMP and di-t-BOC 6.16 g (2.8×10⁻² moles) in 10 ml NMP. Yield 3.3 g (75%).

¹H NMR (DMSO-d₆); δ [ppm]: 6.64-7.09 (36H, aromatic protons), 7.49 (3H, benzylic protons), 5.09 (6H, Ph-CH2), 2.08 (9H, —CH3), 1.47 (40H, —CH3), 9.30 (2H, Ph-OH).

### Example 9

### Preparation of G₁-Tris-t-BOC-60

The compound was prepared by same method used for G₁-Tris-t-BOC100 dendrimer from, 3 g (3×10⁻³ moles) of G₁-Tris and 1.98 g (0.135 moles) DMAP, 25 ml NMP and di-t-BOC 4.62 (2.1×10⁻² moles) in 10 ml NMP. Yield 2.98 g (73%).

¹H NMR (DMSO-d₆); δ [ppm]: 6.65-7.08 (36H, aromatic protons), 7.50 (3H, benzylic protons), 5.10 (6H, Ph-CH2), 2.09 (9H, —CH3), 1.48 (29H, —CH3), 9.25 (2.5H, Ph-OH).

### Example 10

### Preparation of G₁-Tris-t-BOC-50

The compound was prepared by same method used for G₁-Tris-t-BOC-100 dendrimer from, 3 g (3×10⁻³ moles) of G₁-Tris and 1.65 g (0.162 moles) DMAP, 25 ml NMP and di-t-BOC 3.85 (1.75×10⁻² moles) in 10 ml NMP. Yield 2.96 g (76%).

¹H NMR (DMSO-d₆); δ [ppm]: 6.61-7.09 (36H, aromatic protons), 7.49 (3H, benzylic proton), 5.09 (6H, Ph-CH2), 2.08 (9H, —CH3), 1.47 (26H, —CH3), 9.25 (3H, Ph-OH).

### Example 11

### Preparation of (G₁-Bis-t-BOC-100)

The compound was prepared by same method used for G₁-Tris-t-BOC dendrimer from, 3 g (3.75×10⁻³ moles) of G₁-Bis and 2 g (1.65×10⁻² moles) DMAP, 25 ml NMP and di-t-BOC 5 g (2.27×10⁻² moles) in 10 ml NMP. Yield 3.27 g (79%).

¹H NMR (DMSO-d₆); δ [ppm]: 6.90-7.24 (24H, aromatic protons), 7.47 (3H, benzylic protons), 5.08 (6H, Ph-CH2), 1.60 (18H, —CH3), 1.47 (27H, —CH3).

### Example 12

### Preparation of G₁-Bis-t-BOC-80

The compound was prepared by same method used for G₁-Tris-t-BOC dendrimer from, 3 g (3.75×10⁻³ moles) of G₁-Bis and 1.6 g (1.32×10⁻² moles) DMAP, 25 ml NMP and di-t-BOC 4 g (1.81×10⁻² moles) in 10 ml NMP. Yield 2.97 g (76%).

¹H NMR (DMSO-d₆); δ [ppm]: 6.90-7.19 (24H, aromatic protons), 7.47 (3H, benzylic protons), 5.07 (6H, Ph-CH2), 1.60 (18H, —CH3), 1.47 (14 H, —CH3) 9.17 (1.5H, Ph-OH).

### Example 13

### Preparation of G₁-Bis-t-BOC-60

The compound was prepared by same method used for G₁-Tris-t-BOC dendrimer from, 3 g (3.75×10⁻³ moles) of G₁-Bis and 1.2 g (9.9×10⁻³ moles) DMAP, 25 ml NMP and di-t-BOC 3 g (1.36×10⁻² moles) in 10 ml NMP. Yield 2.65 g (72%).

¹H NMR (DMSO-d₆); δ [ppm]: 6.62-7.19 (24H, aromatic protons), 7.47 (3H, benzylic protons), 5.07 (6H, Ph-CH2), 1.55-1.60 (18H, —CH3), 1.47 (11 H, —CH3) 9.17 (1.7H, Ph-OH).

### Example 14

### Preparation of G₁-Bis-t-BOC-50

The compound was prepared by same method used for G₁-Tris-t-BOC dendrimer from, 3 g (3.75×10⁻³ moles) of G₁-Bis and 1g (8.2×10⁻³ moles) DMAP, 25 ml NMP and di-t-BOC 2.5 g (1.13×10⁻² moles) in 10 ml NMP. Yield 2.50 g (70%).

¹H NMR (DMSO-d₆); δ [ppm]: 6.62-7.19 (24H, aromatic protons), 7.47 (3H, benzylic protons), 5.07 (6H, Ph-CH2), 1.55-1.60 (18H, —CH3), 1.46 (9H, —CH3) 9.16 (2H, Ph-OH).

### Example 15

### Preparation of (G₁-Dhn-t-BOC-100)

The compound was prepared by same method used for G₁-Tris-t-BOC dendrimer from, 3 g (4.5×10⁻³ moles) of G₁-Dhn and 2.4 g (1.95×10⁻² moles) DMAP, 25 ml NMP and di-t-BOC 5.85 g (2.7×10⁻² moles) in 10 ml NMP. Yield 3.26 g (72%).

¹H NMR (DMSO-d₆); δ [ppm]: 8.16 (3H, protons on 8th carbon of naphthalene ring), 7.75 (3H, protons on 4th carbon of naphthalene ring), 6.59-7.19 (6H, protons on 2nd and 6th carbon of naphthalene ring), 7.63 (3H, protons on 3rd carbon of naphthalene ring), 7.34 (3H, protons on 7th carbon of naphthalene ring), 7.46 (3H, benzylic protons), 5.43 (6H, Ph-CH2), 1.52 (27H, CH3).

### Example 16

### Preparation of G₁-Dhn-t-BOC-80

The compound was prepared by same method used for G₁-Tris-t-BOC dendrimer from, 3 g (4.5×10⁻³ moles) of G₁-Dhn and 1.92 g (1.56×10⁻³ moles) DMAP, 25 ml NMP and di-t-BOC 4.68 g (2.1×10⁻³ moles) in 10 ml NMP. Yield 2.78 g (66%).

¹H NMR (DMSO-d₆); δ [ppm]: 6.59-8.12 (18H, naphthalene ring protons, same splitting of ring protons was observed as discussed in section 2.3.12), 7.46 (3H, benzylic protons), 5.42 (6H, Ph-CH2), 1.52 (17H, CH3), 10.11 (1H, hydroxyl proton of naphthalene).

### Example 17

### Preparation of G₁-Dhn-t-BOC-60

The compound was prepared by same method used for G₁-Tris-t-BOC dendrimer from, 3 g (4.5×10⁻³ moles) of G₁-Dhn and 1.44 g (1.17×10⁻² moles) DMAP, 25 nil NMP and di-t-BOC 3.57 g (1.6×10⁻² moles) in 10 ml NMP. Yield 2.66 g (67%).

¹H NMR (DMSO-d₆); δ [ppm]: 6.59-8.12 (18H, naphthalene ring protons), 7.46 (3H, benzylic protons), 5.42 (6H, Ph-CH2), 1.52 (14H, CH3), 10.11 (1.54H, hydroxyl protons of naphthalene).

### Example 18

### Preparation of G₁-Dhn-t-BOC-50

The compound was prepared by same method used for G₁-Tris-t-BOC dendrimer from, 3 g (4.5×10⁻³ moles) of G₁-Dhn and 1.2 (9.7×10⁻³ moles) DMAP, 25 ml NMP and di-t-BOC 2.97 g (1.3×10⁻² moles) in 10 ml NMP. Yield 2.44 g (65%).

¹H NMR (DMSO-d₆); δ [ppm]: 6.59-8.12 (18H, naphthalene ring protons), 7.46 (3H, benzylic protons), 5.42 (6H, Ph-CH2), 1.52 (12H, CH3), 10.11 (1.84H, hydroxyl protons of naphthalene).

### Example 20

### Thermal Properties

### Thermal Degradation

Thermal stability of the FGDs was evaluated using TGA-7, Perkin Elmer at a heating rate 10° C./min under nitrogen atmosphere. The temperature Tₛ corresponding to 5 wt % loss was considered as the index of thermal stability (Table 1).

### Example 21

### Glass Transition Temperature

T_{g}s of FGDs were determined using TA Instruments DSC Q-10 in nitrogen atmosphere at a heating rate 10° C./min and the data are presented in Table 1.

### Example 22

### Lithographic Evaluation

The FGDs were dissolved in propylene glycol methyl ether acetate (PGMEA) to obtain a 5 wt. % solution. Commercially available triphenylsulfonium perfluoro-1-butanesulfonate (PAG) 5 wt. % and 10 wt. % on the basis of resist was used. The resulting solutions were filtered through a 200 nm filter and spin coated onto 2 inch oxidised silicon wafers (oxide thickness 200 nm) at 6000 rpm for 30 sec, leading to a film thickness of 66 nm for G₁-Tris-epoxide, 63 nm for G₁-Bis-epoxide and 62 nm for G₁-Dhn-epoxide as measured by Raith-150^{Two} SEM. This was subjected to prebaking at 70° C. for 5 min on hotplate and then exposed to e-beam using Raith-150^{Two}. Post exposure baking was done at 90° C. for 60 sec on a hotplate. The wafers were then developed using propylene glycol methyl ether acetate (SU-8 developer) for 30 sec and rinsed with IPA for 10 sec and dried with a nitrogen blower.

### Example 23

### Sensitivity Curve for the FGDs

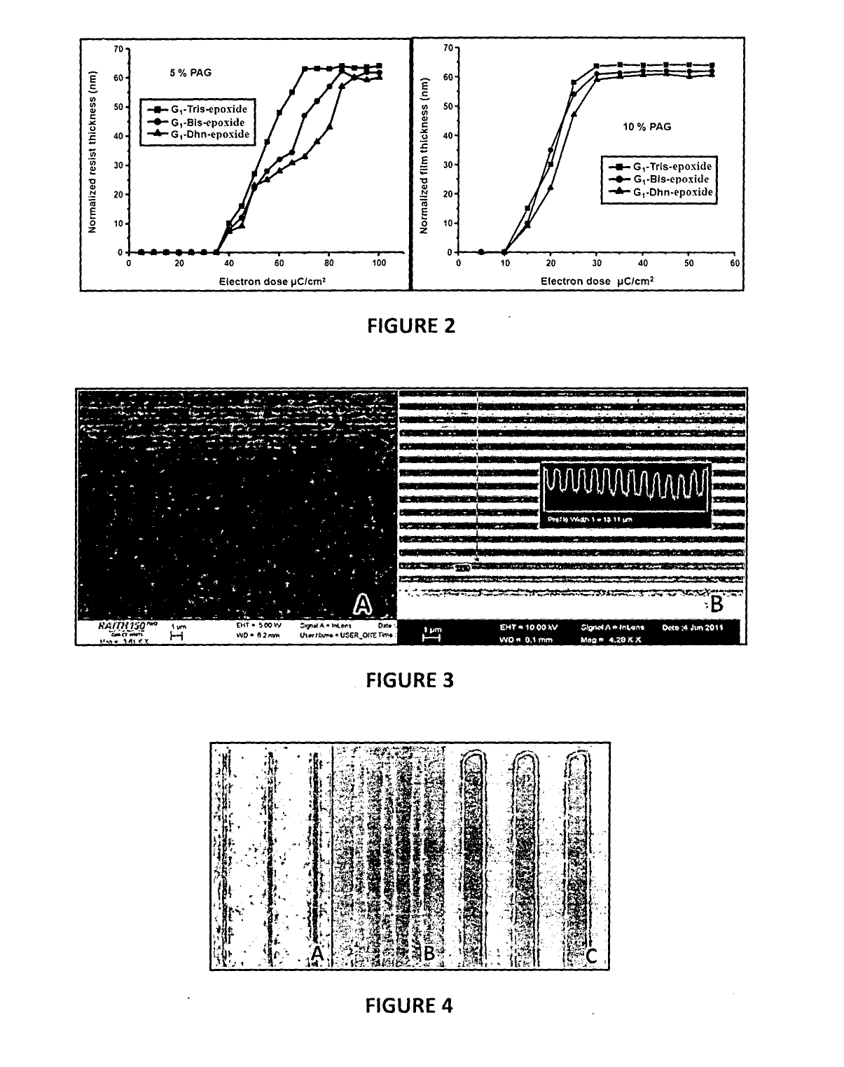

The sensitivity curves for the negative tone resists on exposure to e-beam at 20 kV acceleration voltage, 20 μm aperture, resulting in a beam current of 170 pA are shown in FIG. 2. All the resists containing 5 wt % FGDs and 5 wt. % as well as 10 wt. % PAG on the basis of FGDs in PGMEA were spin coated on a silicon substrate and exposed in the dose range 5 μC/cm² to 100 μC/cm². A series of 500 nm lines were patterned at pitch 1:1 for all the FGDs. Typical patterns for G₁-Tris-epoxide are shown in FIGS. 3A and 3B.

Resist sensitivity was calculated by measuring the thickness of the lines patterned at various dose rates using Raith 150^{Two} SEM. Sensitivity was defined as the dose D1 at which thickness of the developed pattern was the same as that of spin coated film.

The resist containing 5 wt % PAG shows a sensitivity of 70 μC/cm² for G₁-Tris-epoxide, 85 μC/cm² for G₁-Bis-epoxide and 95 μC/cm² for G₁-Dhn-epoxide (FIG. 2). At 10 wt % PAG content all resists showed a sensitivity of 35 μC/cm² (FIG. 2).

The contrast (γ) was calculated from the formula γ⁻¹=log D₁−log D₀ where D₀ is the highest dose where the resist is not crosslinked. D₁ is the dose where the resist height after development is the same as the thickness of spin coated film. The resists show a sensitivity of 35 μC/cm² and lines upto 30 nm and pitch 1:1 could be resolved with high contrast 3.3 at 20 kV. The results are summarized in Table 3.

TABLE 3
Comparison of the FGDs with SU-8

| | Acceleration | Sensitivity | Contrast | Linewidth | Pitch |
|---|---|---|---|---|---|
| Resist | (kV) | (μC/cm²) | (γ) | (nm) | (nm) |
| SU-8 | 50 | 3.6 | — | 75 | |
| SU-8 | 100 | 19.9 | 0.9 | 24 | 300 |
| G₁-Tris-epoxide | 20 | 35 | 3.3 | 30 | 30 |
| G₁-Bis-epoxide | 20 | 35 | 2.78 | 30 | 30 |
| G₁-Dhn-epoxide | 20 | 35 | 2.43 | 50 | 500 |

### Example 24

### Electron Beam Lithography

Oxidised silicon wafers were used to enhance adhesion of photoresist to the substrate. Resists containing 5 wt. % FGDs and 10 wt. % triphenylsuiphonium-nanoflate (PAG) on the basis of FGDs were spin-coated onto two inch silicon wafers at 6000 rpm which formed 66 nm (G₁-Tris-epoxide), 63 nm (G₁-Bis-epoxide) and 62 nm (G₁-Dhn-epoxide) thick films respectively. Soft baking of the coated films on the silicon wafer was carried out on hot plate at 70° C. for 5-min.The resists were patterned using e-beam at 20 kV acceleration voltage, 20 μm aperture and the dose 35 μC/cm²at beam current 170 pA. Post exposure bake (PEB) was carried out at 90° C. for one min. Post exposure baked films were developed with propylene glycol methyl ether acetate (SU-8 developer MicroChem) for 20 sec, followed by a 10 sec immersion in isopropyl alcohol.

### Line Edge Roughness (LER)

LER was calculated using the formula reported by Leunissen et al., (2004) for feature size 100, 50 and 30 nm. FIGS. 4A, 4B and 4C show typical images of 30, 50 and 100 nm lines of G₁-Tris-epoxide, used for LER calculation.

The LER values calculated are based on an average of 20 adjacent points along the lines and are summarized in Table 4.

TABLE 4
LER values comparison

| | Line width | LER | Sensitivity |
|---|---|---|---|
| Resist | (nm) | (3σ) | (μC/cm²) |
| G₁-Tris-epoxide | 100 nm | 5.40 | 35 |
| | 50 nm | 5.80 | (20 KeV) |
| | 30 nm | 6.00 | |
| G₁-Bis-epoxide | 100 nm | 5.63 | 35 |
| | 50 nm | 5.70 | (20 KeV) |
| | 30 nm | 6.30 | |
| G₁-Dhn-epoxid | 100 nm | 6.80 | 35 |
| | 50 nm | 7.80 | (20 KeV) |

### Example 26

### Optical Lithography

The resists containing 14.4 wt % dendrimer (G₁-Tris-epoxide, G₁-Bis-epoxide and G₁-Dhn-epoxide) solutions in PGMEA were used to match the composition of SU-8 (0.5).

Triarylsulphonium hexafluoroantimonate (PAG) which is also used in SU-8 (0.5), 10 wt % on the basis of FGDs was added. SU-8 (0.5) was used as resist for comparison. Standard RCA (Radioactive Corporation of America) cleaned 2-inch silicon wafers were used, which were cleaned by immersion in hydrofluoric acid (HF) and washed with water. Wet oxidation was carried out to grow 490 nm thick silicon dioxide layer. This was followed by spin-coating of resists at 6000 rpm. SU-8 (0.5) was first spin-coated onto the substrate to form resist layer 460 nm thick. The FGDs were spin-coated onto the silicon substrate to form a resist layers 200 nm thick for G₁-Tris-epoxide, 150 nm for G₁-Bis-epoxide and 110 nm for G₁-Dhn-epoxide. Coated samples were processed using a standard procedure which involved pre-baking on a hotplate at 70° C. for 5 min and 90° C. for 1 min, followed by exposure using a soft-contact mask-aligner of 60 mJ/cm² intensity at 365 nm wavelength for 5 sec. Post exposure bake (PEB) was carried out at 60° C. for 1 min and 95° C. for 1 min to accelerate cross linking of the exposed areas of the photoresist. The patterns were developed using PGMEA (SU-8 developer) at room temperature rinsed with isopropanol and dried with a nitrogen blower.

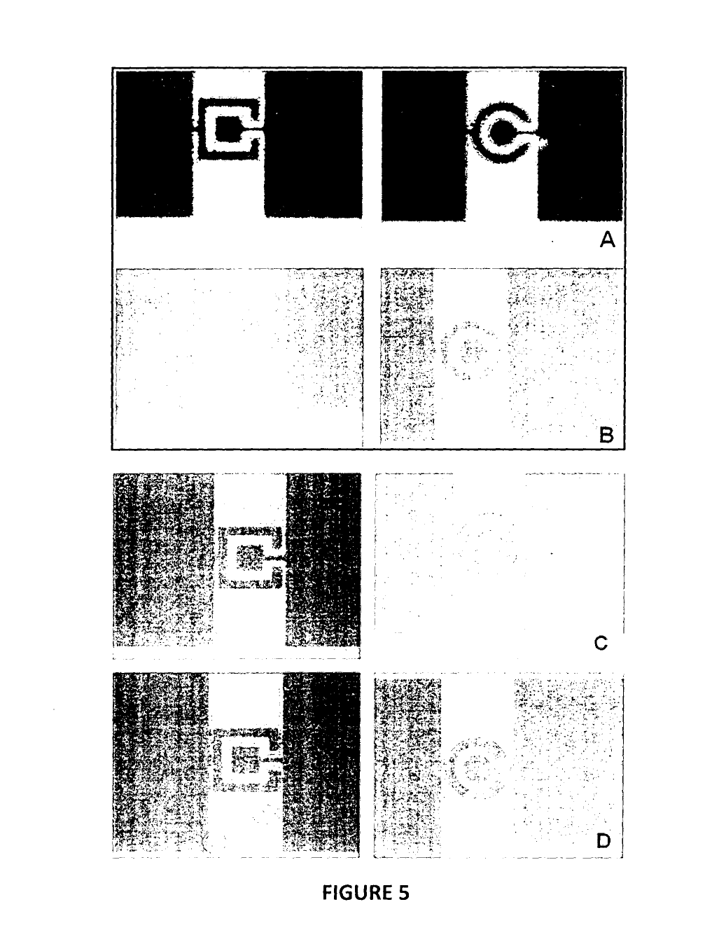

FIG. 5 shows colored optical images of the SU-8 (FIG. 5a) and FGDs G₁-Tris-epoxide (FIG. 5b), G₁-Bis-epoxide (FIG. 5c) and G₁-Dhn-epoxide (FIG. 5d) resists respectively.

The FGDs based on trisphenol and bisphenol could resolve defect free features after development.

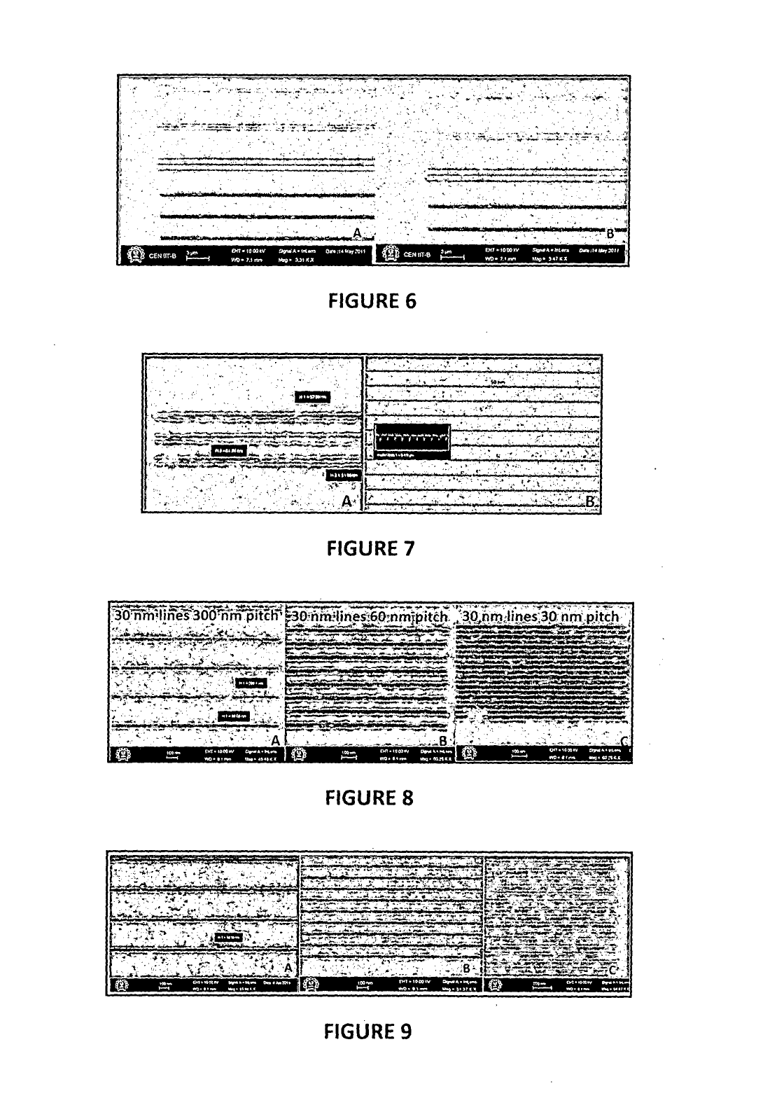

FIGS. 6 (A) and (B) show scanning electron micrographs for 500, 200, 100 and 50 nm patterns for G₁-Tris-epoxide and G₁-Bis-epoxide using same mask. At this magnification 50 nm lines were not seen. At higher magnification 50 nm lines at 1:2 nm pitch were resolved (FIG. 7 A). However in the case of G₁-Dhn-epoxide 50 nm lines could be resolved at1:10 pitch (FIG. 7 B).

FIGS. 8 and 9 show pattern images of FGDs based on trisphenol and bisphenol-A respectively. SEM demonstrates that 30 nm lines could be resolved when the pitch was 1:10

(A), 1:2 (B), and 1:1(C). Thus trisphenol as well as bisphenol based FGDs could be used to pattern features upto 30 nm at the pitch 1:1.

### Example 27

### Reactive Ion Etching (RIE)

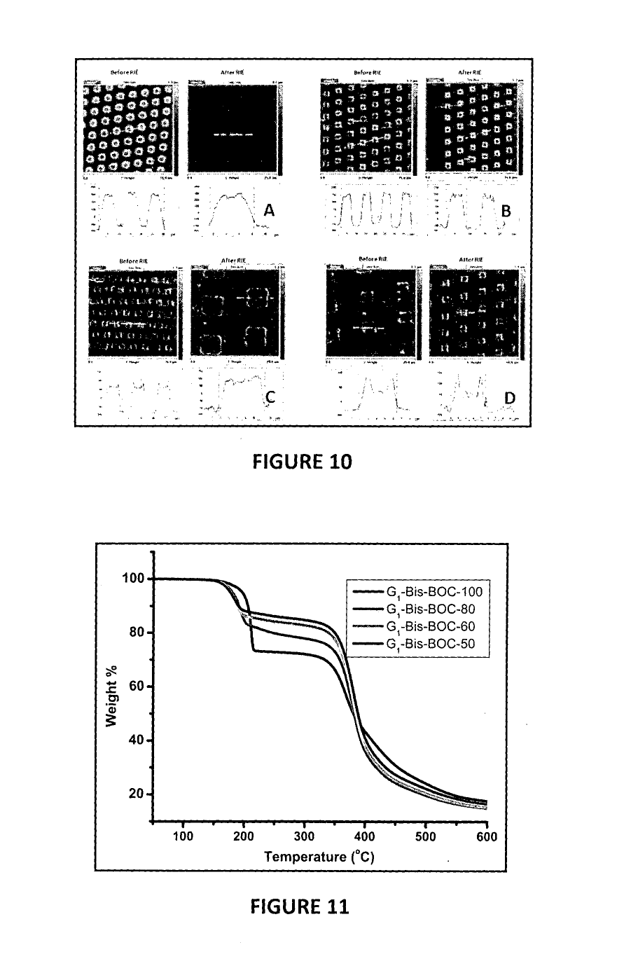

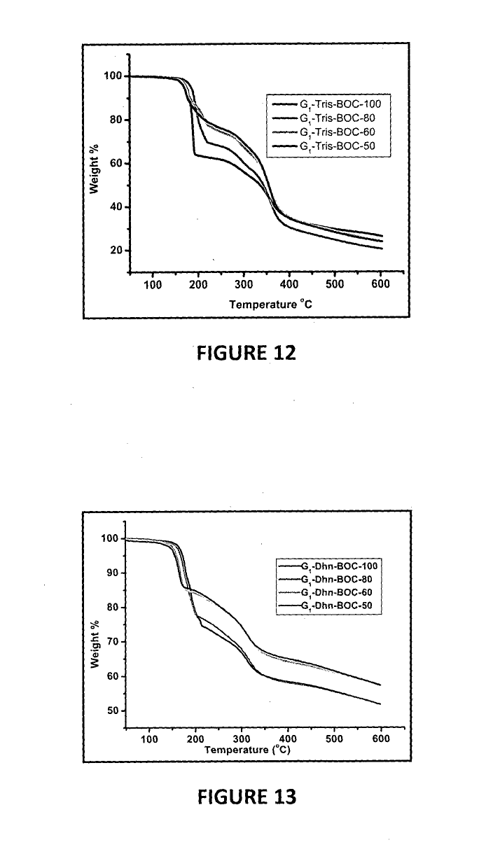

To transfer the resist pattern into silicon, reactive ion etching process was conducted. The process was carried out for five minutes. Etch rates of resist and SiO₂ was measured using SEM by measuring the resist thickness. In addition, etch rates of these resists were also examined with a standard AFM. Thickness of the oxide layer plus resist layer (C) was measured by SEM. The etch rate was calculated by the difference of the thickness before (C) and after (F) etching as shown in FIG. 10.

The etch rate in resist and silicon oxide are described in Table 5

The RIE results were compared with SU-8.

TABLE 5
Reactive ion etching (RIE) of the FGDs and SU-8

| | | B | | | | | G | H |
|---|---|---|---|---|---|---|---|---|
| Name | A (nm) | (nm) | C (nm) | D (nm) | E (nm) | F (nm) | (nm/sec) | (nm/sec) |
| G₁-Tris-ep | 200 | 490 | 690 | 270 | 350 | 620 | 0.23 | 0.46 |
| G₁-Bis-ep | 150 | 490 | 640 | 200 | 350 | 550 | 0.30 | 0.46 |
| G₁-Dhn- | 110 | 490 | 600 | 180 | 350 | 530 | 0.23 | 0.46 |
| ep | | | | | | | | |
| SU-8 | 460 | 490 | 950 | 500 | 350 | 850 | 0.33 | 0.46 |

A=Resist thickness before RIE (AFM measurements); B=Dioxide thickness before RIE (SEM measurements); C=Total thickness before RIE (A+B); D=Thickness after RIE (resist+dioxide) (AFM measurements); E=Dioxide thickness after RIE (SEM measurements); F=Total thickness after RIE (D+E); G=Resist etching rate (C−F); H=Dioxide etching rate (B−E).

### Example 28

### Thermal Degradation Positive Resist

Dendrimers and their t-BOC conjugates were evaluated usingTGA-7, Perkin Elmer at 10° C./min under nitrogen atmosphere. The plots in the FIGS. 11 (G₁-Bis-t-BOC), 12 (G₁-Tris-t-BOC) and 13 (G₁-Dhn-t-BOC) show two step degradation profile attributed to the deblocking of t-BOC in the range 160 to 210° C. followed by degradation beyond 250° C. The weight loss in the temperature range 160° C. to 210° C. corresponds to the loss of t-BOC group in the form of carbon monoxide and isobutylene. t-BOC deblocking temperature for 50% t-BOC conjugated FGDs were G₁-Bis-t-BOC (176° C.), G₁-Tris-t-BOC (168° C.) and G₁Dhn-t-BOC (155° C.) respectively.

### Example 30

### The Glass Transition Temperature

T_{g}s of dendrimers were determined by TA Instruments DSC Q-10 in nitrogen atmosphere at a heating rate 5° C./min and the data are presented in Table-6. The traces indicate no melting peak confirming amorphous nature of the materials.

TABLE 6
DSC analysis of t-BOC FGDs

| No. | FGDs | T_{g}° C. |
|---|---|---|
| 1 | G₁-Tris-t-BOC100 | 119 |
| 2 | G₁-Tris-t-BOC80 | 125 |
| 3 | G₁-Tris-t-BOC60 | 130 |
| 4 | G₁-Tris-t-BOC50 | 128 |
| 5 | G₁-Tris | 139 |
| 6 | G₁-Bis-t-BOC100 | 45 |
| 7 | G₁-Bis-t-BOC80 | 58 |
| 8 | G₁-Bis-t-BOC60 | 63 |
| 9 | G₁-Bis-t-BOC50 | 68 |
| 10 | G₁-Bis | 70 |
| 11 | G₁-Dhn-t-BOC100 | 126 |
| 12 | G₁-Dhn-t-BOC80 | 118 |
| 13 | G₁-Dhn-t-BOC60 | 116 |
| 14 | G₁-Dhn-t-BOC50 | 116 |
| 15 | G₁-Dhn | 87 |

### Example 31

### Adhesion Performance:

Adhesion of polymers on the silicon substrate was evaluated by calculating work of adhesion (W_{ad}) from the following eqn.

W_{ad}=2{(γp^{d}·γs^{d})^{1/2}+(γp^{h}·γs^{h})^{1/2}} (1)

(Where γ: surface free energy, P: polymer, S: substrate, d: dispersion force, h: hydrogen bonding force). To solve this equation, the contact angles (θ) of water and diiodomethane were measured. The values of contact angle and work of adhesion for various t-BOCconjugated FGDs films on silicon wafer are given in Table-7.

TABLE 7
Contact angle and work of adhesion for various t-BOC films to silicon substrate

| Dendrimers | G₁-Tris-t-BOC | | | | G₁-Bis-t-BOC | | | | G₁-Dhn-t-BOC | | | | Si |
|---|---|---|---|---|---|---|---|---|---|---|---|---|---|
| A | 50 | 60 | 80 | 100 | 50 | 60 | 80 | 100 | 50 | 60 | 80 | 100 | — |
| θ (H₂O) | 88 | 89.2 | 78.9 | 96.9 | 72.9 | 73 | 78.9 | 90.3 | 74.8 | 82.5 | 81.1 | 87.8 | 39.8 |
| θ (CH₂I₂) | 32 | 28.7 | 27 | 25 | 29.3 | 20 | 15.6 | 16.3 | 43 | 39.9 | 31.2 | 27.3 | 28.2 |
| W_{ad} | 59.4 | 55.5 | 42.3 | 39.3 | 82 | 79 | 68.8 | 48.6 | 83.4 | 71.8 | 70.7 | 57 | — |

A = t-BOC (mole %)
θ = Contact angle,
W_{ad} = work of adhesion (W_{ad}/dyne cm⁻¹),

### Example

### Lithographic Evaluation of FGDs as Positive Tone Resists

The FGDs were dissolved in propylene glycol methyl ether acetate (PGMEA) making a 5 wt % solution. Commercially available triphenylsulfonium perfluoro-1-butanesulfonate (10 wt. % with respect to resist) was used as PAG. The resulting solutions were filtered through a 0.2 μm filter. Then, the solutions were spin coated onto a 2 inch silicon wafers at 6000 rpm, for 30 s. This was prebake at 70° C. for 5 min, and then exposed using e-beam radiation. After exposure, the wafer was baked at 90° C. for 60 seconds. Positive tone images were developed in an aqueous solution developer (0.26 N TMAH in DI water) for 80 seconds.

### Example 32

### Sensitivity Curve for the Dendrimeric Positive Resist

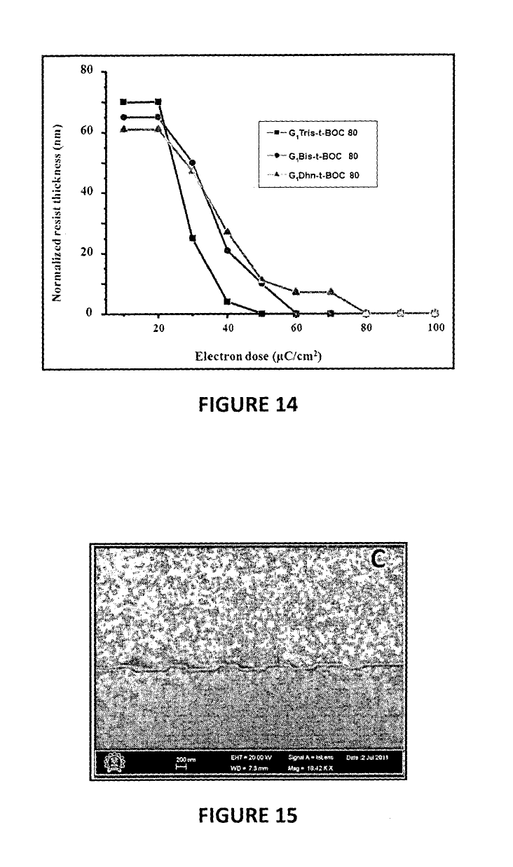

The sensitivity curves for the positive-tone dendrimeric resists on exposure to electron beam at 20 keV acceleration voltage, 20 μm aperture, resulting in a beam current of 157 pA (Pico ampere) are shown in FIG. 14. 80% t-BOC conjugated FGDs resist containing 5 wt % FGD and 10 wt. % PAG (weight of PAG with respect to the weight of dendrimer) in PGMEA spin-coated on silicon substrate were exposed in the dose range from 20 μC/cm² to 200 μC/cm² with the largest beam current 157 pA. The pattern used was a series of 500 nm lines with 500 nm pitch. Resist sensitivity was calculated by measuring cross-section of the lines drawn at various dose rates using Raith-150^{two} SEM (FIG. 15). Sensitivity values were defined as the minimum dose D1 at which the resist was completely washed out after development.

Resists containing 10 wt % PAG showed higher sensitivity 50 μC/cm² (G₁-Tris-t-BOC 80), 60 μC/cm² (G₁-Bis-t-BOC 80) and 80 μC/cm² (G₁-Dhn-t-BOC 80) at 20 keV electron beam acceleration. The contrast (γ) was calculated by using formula (γ¹=log D₁−log D₀), where D₀ is the maximum dose where the resist thickness before development is the same as after development D₁ is minimum dose where the resist is washed out after development. Higher contrast obtained using 10 wt % PAG is γ=2.50 (G₁-Tris-t-BOC 80), γ=2.09 (G₁-Bis-t-BOC 80), γ=1.66 (G₁-Dhn-t-BOC 80).

### Example 33

### Electron Beam Lithography

The evaluation of the system for EBL resist was performed using FGDs with 80% t-BOC conjugation. Oxidised silicon wafer was used to enhance adhesion of photoresist to the substrate.A 5 wt. % solution of 80% t-BOC conjugated FGDs (G₁-Tris-t-BOC, G₁-Bis-t-BOC, G₁-Dhn-t-BOC) and 10 wt. % (with respect to dendrimer) PAG (triphenylsulphoniumnanoflate) was spin-coated onto a two inch silicon wafer at a 6000 rpm which form 70 nm, 64 and 61 nm thick films respectively. Prebaking of the coated film on the silicon wafer was carried out at 70° C. for 5-minutes. Positive-tone FGD systems were tested using e-beam exposure 20 kV at a dose 50 μC/cm², 60 μtC/cm²and 80 μC/cm² respectively. The post exposure bake (PEB) process, carried out at 90° C. for one minute. Post exposure baked film was developed with 0.26N TMAH for 85 seconds.

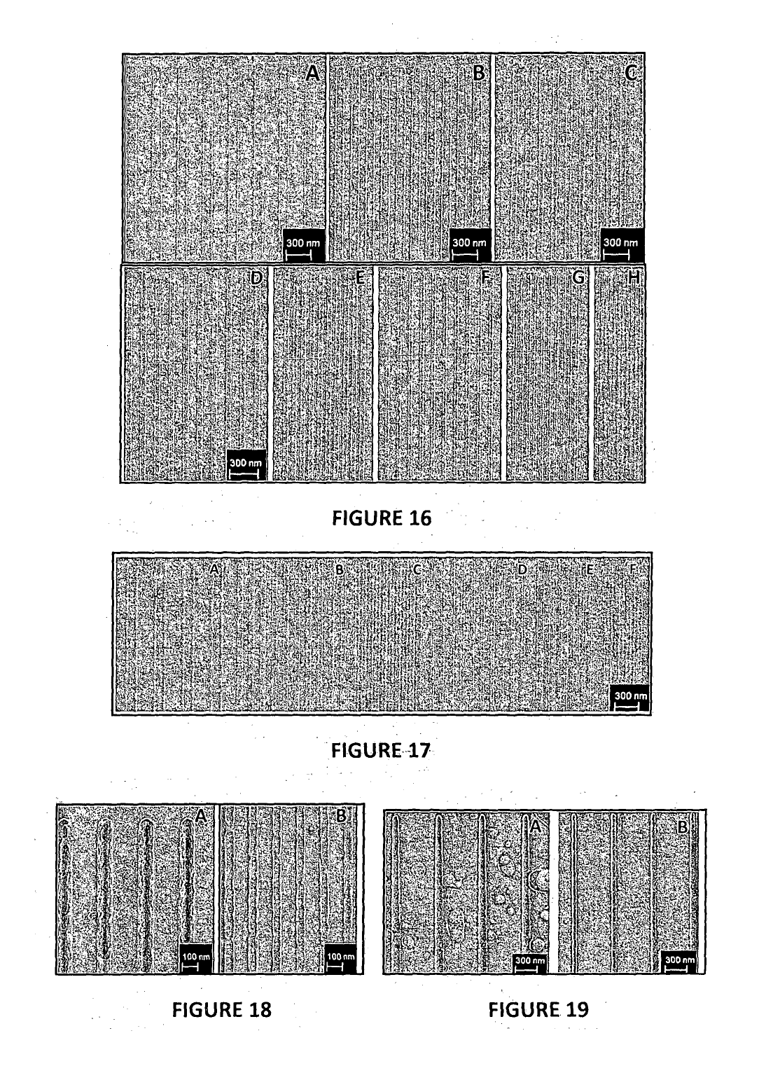

FIGS. 16, 17 and 18 show SEM images of 80% t-BOC conjugated G₁-Tris-t-BOC, G₁-Bis-t-BOC and G₁-Dhn-t-BOC positive tone dendrimers respectively. SEM images demonstrate that 30 nm resolution was achieved using Raith-150^{Two} EBL, with a resist formulation consisting of 5 wt % dendrimer and photoacid generator (triphenylsulphonium2-(phenoxy)tetrafluoroethane-1-sulfonate) with exposure dose of 50, 60 and 80 μC/cm²and 20 kV acceleration, and the pitch was set to 1:1.

FIG. 16 shows SEM images of the FGD based on trisphenol demonstrate 100 nm lines with pitch 1:2 (FIG. 16 A), 1:1 (FIG. 16 B), 1:0.7 (FIG. 16 C), 50 nm lines with pitch 1:2 (FIG. 16D), 1:1 (FIG. 16E) and 30 nm lines 1:3 (FIG. 16 F), 1:2 (FIG. 16G) and 1:1 (FIG. 16 H) respectively. Similar pattern was obtained with FGD based on bisphenol FIG. 17. Whereas FGD based on naphthalene resolved features upto 100 and 50 nm with 1:2 pitch (FIG. 18 A and B). The best resolution was obtained using a PEB at 90° C. for 60 seconds and 70 second development. Compared to all three FGDs, the results obtained using G₁-tris-t-BOC as a positive resist gave better resolution below 50 nm line space with low line edge roughness (LER).

### Line Edge Roughness (LER)

Measured LER values are given in Table-8.

TABLE 8
The measured values of LER for varing features
size of different FGDs resists.

| | | | Sensitivity | |
|---|---|---|---|---|
| | Line width | LER | (μC/cm²) | Contrast |
| Resist | (nm) | (3σ) | At 20 kV | (γ) |
| G₁-Tris-t-BOC80 | 100 nm | 4.9 | 50 | 2.5 |
| | 50 nm | 5.3 | | |
| | 30 nm | 5.8 | | |
| G₁-Bis-t-BOC80 | 100 nm | 5.1 | 60 | 2.09 |
| | 50 nm | 5.7 | | |
| | 30 nm | 6.0 | | |
| G₁-Dhn-t-BOC 80 | 100 nm | 7.6 | 80 | 1.66 |
| | 50 nm | 7.9 | | |

### Example 34

### Negative-Tone Resists

FIG. 19 shows SEM images of the FGD based on trisphenol demonstrate 100 and 50 nm lines with pitch 1:5 (FIG. 27A) and 1:10 (FIG. 27B). Every step is similar for negative and positive dendrimers except developer. Negative tone dendrimers were developed in DCM.

1. First generation dendrimers comprising 1,3,5-trisbromo-methylbenzene as the core and dense, bulky, rigid units selected from Trisphenol (1, 1, 1-tris-p-4-hydroxyphenyl ethane), bisphenol-A and 1,5-dihydroxy naphthalene units at the periphery, wherein the peripheral aromatic rigid molecules are connected to the central core through an ether linkage.

2. The first generation dendrimers according to claim 1, wherein the dendrimers based on trisphenol (1, 1, 1-tris-p-4-hydroxyphenyl ethane) comprise six hydroxyls at the periphery.

3. The first generation dendrimers according to claim 1, wherein the dendrimers based on bisphenol-A and 1,5-dihydroxy naphthalene comprise three hydroxyl at the periphery.

4. The first generation dendrimers according to claim 1, wherein the dendrimers are further conjugated with either epoxide or with tert-BOC to obtain negative or positive photoresists respectively.

5. The first generation dendrimers according to claim 4, wherein the negative photoresists are selected from the group consisting of G₁-Tris-epoxide, G₁-Bis-epoxide and G1-Dhn-epoxide.

6. The first generation dendrimers according to claim 4, wherein positive photoresists are selected from the group consisting of G₁-Tris-t-BOC-100, G₁-Tris-t-BOC-80, G₁-Tris-t-BOC-60, G₁-Tris-t-BOC-50, G₁-Bis-t-BOC-100, G₁-Bis-t-BOC-80, G₁-Bis-t-BOC-60, G₁-Bis-t-BOC-50, G₁-Dhn-t-BOC-100, G₁-Dhn-t-BOC-80, G₁-Dhn-t-BOC-60 and G₁-Dhn-t-BOC-50.

7. The first generation dendrimers according to claim 6, wherein the yield of positive photoresist is obtained in order of ≧65%.

8. The first generation dendrimers according to claim 6, wherein glass transition temperature of positive photoresists is in the range of 45° C. to 130° C.

9. The first generation dendrimers according to claim 6, wherein the molecular weights of the positive photoresists are in the range of 700 to 1700.

10. The first generation dendrimers according to claim 6, wherein (G₁-Tris-t-BOC 80), (G₁-Bis-t-BOC 80) and (G₁-Dhn-t-BOC 80) having contrast γ=2.50, γ=2.09 and γ=1.66 respectively in presence of 10 wt % PAG.

11. The first generation dendrimers according to claim 6, wherein (G₁-Tris-t-BOC 80), (G₁-Bis-t-BOC 80) and (G₁-Dhn-t-BOC 80) having sensitivity 50 μC/cm²; 60 μC/cm² and 80 μC/cm² respectively at 20 keV electron beam acceleration.

12. A process for synthesis of negative photoresists according to claim 3, comprises reacting phenols/naphthols with 1,3,5-tris-bromomethylbenzene in the presence of potassium carbonate in DMF; conjugating the peripheral hydroxyls with epichlorohydrin using KOH as a base and PEG-400 as a phase transfer catalyst.

13. The process according to claim 11, wherein the molecular weights of the negative photoresists are in the range of 750-1400.

14. The process according to claim 11, wherein the glass transition temperature of negative photoresists is in the range of 60° C. to 90° C.

15. The process according to claim 11, wherein the yield of negative photoresist is obtained in the order of >90%.

16. The process according to claim 11, wherein the negative photoresists with 10 wt % PAG content shows a sensitivity of 35 μC/cm².

17. The process according to claim 11, wherein negative photoresists having resist etching rate in the range of 0.23 to 0.30.
